(19) United States
(12) Patent Application Publication
(10) Pub. No.: US 2017/0232160 A1
(43) Pub. Date: Aug. 17, 2017

(54) INCISION SYRINGE WITH DISPOSABLE CARTRIDGE

(71) Applicant: Gabriel Cardwell, Vista, CA (US)

(72) Inventor: Gabriel Cardwell, Vista, CA (US)

(21) Appl. No.: 15/435,237

(22) Filed: Feb. 16, 2017

Related U.S. Application Data
(60) Provisional application No. 62/295,952, filed on Feb. 16, 2016.

(51) Int. Cl. A61M 1/00; A61M 5/31; A61M 5/315; A61M 5/32; A61B 17/3209; A61B 17/34

(52) U.S. Cl. CPC A61M 1/0009; A61B 17/32093; A61B 17/34; A61M 5/315; A61M 5/3202; A61M 5/3137; A61B 2017/00561

(57) ABSTRACT

An incising and medicating syringe is described which is configured to form raised skin areas of effected skin and lance the skin to extract subdermal fluid and distribute medical ointment. The device is employed by placement of an aperture over the effective area whereafter translation of a plunger extends a lance which subsequently withdraws to absorb and contain the excreted fluid.

### FIELD OF THE INVENTION

This application claims priority to U.S. Provisional Patent Application Ser. No. 62/295952, filed on Feb. 16, 2016, which is incorporated herein in its entirety by this reference thereto.

The present device relates to personal hygiene devices and medical equipment. More particularly, the disclosed device relates to a portable device that is configured to effectively and safely extract pus or other fluids from beneath the skin through incision and medication.

### BACKGROUND OF THE INVENTION

In the field of dermatology, humans and animals often exhibit sub-skin cavities of blood, pus or other fluids, which form abscesses, blisters, carbuncles, pustules, or pimples. Such wounds or inflictions, if not remedied over time by the natural defenses of the body, are treated and generally cured only through lancing, which is generally accomplished using a medical instrument capable of incision or puncture of the skin and any underlying cyst, to provide draining and release the fluid within.

Many such skin lesions and infections, for example pimples, may be self-treated by pinching the effected skin surrounding the infection. While not recommended by some medical professionals, due to the potential for spreading an infection, this pinching action imparts pressure to the underlying skin area. In many instances, this underlying area becomes fluid-filled from a natural body defensive reaction caused by blood ceils of the body attacking bacteria or a pathogen.

By communicating pressure to the skin area, a sealed pore or formed cyst, which may have formed to surround the fluid build up, can be forced to release the fluid. However, imparting pressure in this manner, in addition to the potential of spreading the infection by forcing fluid out of the area, can also injure cells of the skin and underlying tissue. Further, a bursting pore or cyst can eject infected fluid which may spread to other areas of the skin.

Still further, many self-treating patients forget that their hands are home to bacteria and other pathogens and attempt this pressure exertion with unsanitary skin and fingers. Such contact can communicate this bacteria from the fingers to the skin which can cause increased infection. Additionally, imparting pressure to extract infection fluid can injure connective tissues in the areas being pressured and cause swelling, itchiness, pain and subsequent sensitivity.

Scalpels and needles, which can provide means for lancing the infected area can effectively provide a means for extraction and fluid release to treat such ailments. However, such medical instruments in the wrong hands, can cause injury. This is because the untrained user can easily make too large an incision or pierce the flesh to a level deeper than necessary with a needle syringe. Such can carry the infection into the muscles and connective tissues under the skin. Further, the risk of infection from multiple sequential uses of the same needle or the like can cause serious infection. Thus, such devices are not recommended for home treatment.

As such, there is a current unmet need for a device to safely treat pimples, and the like, by effectively puncturing the skin at the specific effected area to form a path for release of infectious fluid. Such a device should be capable of sequential use in single or subsequent treatments but in a manner without the risk of communicating infection from one puncture to subsequent needle punctures. Such a device should contain a sealed retracting and extending lance such as a scalpel or hollow needle, which may be safely employed by non medical professionals to lance an extremely short distance into the skin. Further such a device should include and be adapted to dispense sanitizing and healing medical ointment or fluid.

The forgoing examples of related art and limitations related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### OBJECTS OF TIE INVENTION

It is an object of the present invention to provide a device capable of cleanly and effectively piercing the skin to thereby form a path for the exit of infectious fluids.

It is an additional object of this invention to provide a device with a reusable actuator body, which is easily coupled with a disposable component having a lance or syringe thereby reducing cost and enhancing treatment, while guarantying ongoing sterility.

It is a further object of this invention for the device to be capable of containing and dosing medicated ointment or fluid.

It is yet an additional object of this invention for the device to absorb excreted fluid within a disposable cavity housing the lance or needle.

These and other objects, features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### SUMMARY OF THE INVENTION

In accordance with the purposes of the present invention, as embodied and broadly described herein, the present invention provides a device configured in a particularly preferred mode, as an incising and medicating syringe or lance. The device includes a syringe type barrel which houses internal treatment actuation mechanisms. The device includes means for coupling and decoupling a removably engageable disposable treatment tool, and acts as a grip during use.

In all modes of the device, the barrel should have a diameter and length which are sufficient to house the components housed therein and form a comfortable grip for the human hand.

The barrel should also contain two or more perpendicular flanges at its upper end as finger holds for leverage and stability during treatment. The bottom end of the barrel should additionally contain one or a combination of temporary and lightly sealing means to securely engage the exterior faces of the cap including but not limited to: threads, press fits, or snaps.

The cap includes upper and outer faces and is adapted to removably engage with the barrel through the aforementioned mating and temporary sealing means. The cap is configured to protect the contents of the barrel therein during transportation and storage. One or a combination of relatively air tight but impermanent affixation means including but not limited to: press fits or snaps, should also be employed within the interior surface of the cap to adhere to the outer face or hub.

The fastening means, described above, to connect the hub to the cap and the cap to the barrel should be configured such that each pre-conjoined hub and cap will securely couple to the barrel to perform a single treatment. Thereafter, the cap may be decoupled to be discarded.

The barrel is configured with fastening means capable of enduring multiple coupling and decoupling repetitions of the removably engageable caps. Such will insure device longevity.

The hub includes a radial array of two or more small vent holes to permit airflow through the cap. This cap may optionally include a volume of medical ointment or cream to be dispensed from the device by the plunger during treatment, although the device performs in excellent fashion without this option.

A disposable micro lance, embedded within an axial cavity within the cap, is adapted during activation to extend into the cavity within the cap, or slightly below a lower face of the cap when actuated. The micro lance is sized and sharpened to properly pierce the skin of a user during treatment and minimize any damage.

For increased treatment efficacy and range of uses, the lower face of the cap may contain an angled aperture with a diameter and chamfer capable of effectively surrounding an area or pinching the akin area of the incision area. The aperture may be sized to encourage skin to raise into the cavity of the aperture, in which case, the translation of the micro lance does not need to extend out of the cap.

A containment film, which is shielded by a removable upper sanitary film, maintains sterility of the micro lance during transport and should cover the upper edge of the cap to retain the medical ointment within prior to being pierced by the plunger.

Additionally, the angled aperture of the cap is covered by a removable lower sanitary film to maintain sterility and safety. A push knob contains a female threaded hole on its lower face to fasten to the corresponding mating threads upon the plunger and is constrained to slide axially within the barrel.

The push knob can alternatively fasten to the plunger through one or more of the following rigid and permanent means including but not limited to: magnets, press fit, adhesive, or a flexible coupling, or the push knob and plunger can potentially be combined into a singular component.

The plunger additionally contains one or more O-rings around its outer face, which are sized and assembled to maintain an airtight seal within the barrel, and exhibits a beveled lower face capable of mating with the interior face of the hub.

A captive washer is permanently affixed within the barrel through one or a combination of means including but not limited to: snaps, threads, epoxy, overmo456ulding or press fit. Employing a captive washer increases device durability and reliability by distributing the force of the upper compression spring against the thumb knob as it repeatedly contracts and expands.

Additionally, a lower compression spring, provides a constant force between the lower edge of the cap and the flange on the hub. Both the upper and lower compression springs should preferably have closed and grounded ends to reduce wear against its mating components. The cap, hub, washer, and barrel may also contain proper spring interfaces to prevent rotation of the upper and lower compression springs and reduce wear.

The upper and lower springs can alternatively be replaced by one or a combination of flexing or biasing mechanisms including but not limited to: air springs, leaf springs, disk springs or flexures.

Employing the device described herein, the user removes the upper sanitary film from the cap, affixes the adjoined cap and hub to the bottom opening of the barrel, and removes the lower sanitary film.

The user then firmly grabs the outer cylindrical face of the barrel with one hand and presses the lower angled aperture of the cap firmly against the skin. If the aperture is formed in a diameter of substantially 4 mm, it has been found to allow the skin being contacted by the cap to rise into the axial cavity with which the aperture communicates. This is especially preferred in that it allows the micro lance to be maintained within the cap during use.

The lance, such as a micro scalpel or hollow needle positioned within the cap, may then be actuated by the syringe to translate and lance or pierce the skin in the effected area. This translation of the lance as noted, may be entirely inside the cap, where the aperture is formed with a diameter which allows skin to rise into the axial cavity adjacent the aperture.

Actuation is accomplished when the user presses one finger of his or her other hand against the bottom face of each of the perpendicular flanges on the barrel for leverage, and then depresses the thumb knob. Depressing the thumb knob collapses the upper compression spring and drives the plunger down through the barrel to pierce the containment film and expel any ointment through the air vents of the hub.

The bevel of the plunger then couples with and unseats the hub from its temporary connection to the cap, collapses the lower compression spring, and translates the hub to the bottom of the cap. This translates the micro lance toward the aperture in the lower end of the cap and either pierces the skin drawn into the cavity behind the aperture, or the micro lance may translate to slightly protrude from the aperture in the cap to pierce the effected skin. It is preferred that the micro lance contact the skin within the axial cavity adjacent the aperture.

Once user pressure is removed from the thumb knob, the upper and lower compression springs expand to bias the thumb knob and plunger and thereby translate them within the barrel in a direction toward the plunger.

As an air tight interface exists between the inner wall of the barrel and the O-ring of the plunger, the post-incision vertical translation of the plunger may be configured to communication suction to the incision site, and draw extra ointment, pus, blood or other fluid from the effected area into the cap for easy disposal.

In order to maintain sterility with each successive use, the user discards each used cap after a single use. For a subsequent use, a new sealed cap is engaged. It is recommended that the patient clean the affected area before and after treatment with an antibacterial or alcohol to prevent infection.

In another mode of the device, the hub may be rigidly and temporarily coupled directly to the plunger, eliminating the requirement for a lower compression spring and any films. This mode, however, does not allow the device to contain and emit any ointment or to hold any post-incision fluid. The hub should preferably affix to the plunger through matching threads, but can alternatively employ one or a combination of rigid and temporary fastening means including but not limited to: snaps, press fits or keyed grooves.

The cap, which as noted, is configured to protect the user from the micro lance during transport, and in the preferred mode maintain the micro lance within an axial cavity during use, maintains product sterility as per the first preferred mode of the device. The cap and hub may also couple through an air tight press fit, whereas three to ten mating faces, with a convex polygenic cross section, allows a user to rotate the hub within the barrel.

The hub and cap can alternatively engage through one or a combination of air tight reversible means including but not limited to: threads, snaps or keyed grooves. The cap, in this second preferred mode of the device, should extend through and past the bottom aperture in the barrel to allow the user to engage the hub to the plunger via the cap while the device remains in its expanded configuration.

Employing the device herein per its second configuration, the user inserts the preassembled and sealed hub and cap assembly through the lower aperture in the barrel, affixes the hub to the plunger via the cap, and removes the cap.

After proceeding with treatment as described above in the first preferred mode of the device, the user employs the cap to decouple the hub from the plunger and can then discard the cap and hub.

The barrel, thumb knob, plunger, cap, and hub can be composed of one or a combination of medical grade and preferably transparent materials including but not limited to:

polycarbonate, acrylic, plastic or glass.

One or a combination of non-reactive, medical grade and plastically deforming materials can be used to construct the upper and lower springs including but not limited to: steel, stainless steel, aluminum, plastic, carbon fiber, or wood.

The containment film, upper sanitary film, and lower sanitary film can be composed of any materials which are typically used as thin covering materials, and may contain medical grade adhesive backing for adhesion to their respective components.

The captive washer can be made of one or a combination of durable medical grade materials including but not limited to: plastic, aluminum, steel, stainless steel, polycarbonate, or fiberglass.

The o-rings can be composed of any non-reactive medical grade materials commonly used to make o-rings including but not limited to: silicone or rubber.

With respect to the above description, before explaining at least one preferred embodiment of the herein disclosed invention in detail, it is to be understood that the invention is not limited in its application to the details of construction and to the arrangement of the components in the following description or illustrated in the drawings.

The device herein described and disclosed in the various modes and combinations is also capable of other embodiments and of being practiced and carried out in various ways which will be obvious to those skilled in the art. Any such alternative configuration as would occur to those skilled in the art is considered within the scope of this patent. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other powder and pill storage and dispensing products for carrying out the several purposes of the present disclosed device. It is important, therefore, that the claims be regarded as including such equivalent construction and methodology insofar as they do not depart from the spirit and scope of the present invention.

### BRIEF DESCRIPTION OF DRAWING FIGURES

The accompanying drawings, which are incorporated herein and form a part of the specification, illustrate some, but not the only nor exclusive examples of embodiments and/or features of the disclosed device. It is intended that the embodiments and figures disclosed herein are to be considered illustrative of the invention herein, rather than limiting in any fashion.

In the drawings:

FIG. 1 depicts a front crossed section view of the device actuator engaged with a cap in the primed or retracted configuration in one preferred mode of the device.

FIG. 2 displays a second front crossed section view of the device showing it in the extended or treatment configuration of preferred mode of FIG. 1.

FIG. 3 shows an exploded view of the device of FIGS. 1-2 showing the components thereof.

FIG. 4 depicts a front crossed section view of the retracted position of another preferred mode of the device engaged with an elongated cap.

FIG. 5 displays a front crossed, section view of the extended or treatment position of the device of FIG. 4.

FIG. 6 shows an expanded front view of the device of FIGS. 4-5.

FIG. 7 depicts another mode of the device having an actuator within the axial passage of the barrel in the retracted position and shows the removably engageable cap having a lance biased and translated away from the aperture.

FIG. 8 shows the device of FIG. 7 moved to the extended position with the plunger of the actuator translated toward the removable cap a distance to move the lance engaged with the hub toward but not extending from the aperture.

FIG. 9 shows an exploded view of the device of FIGS. 7-8.

FIG. 10 depicts the removably engageable cap housing the biased lance within, having a film seal located on both ends to maintain sterility within the interior chamber of the cap.

FIG. 11 shows a first side of the hub which is slidably engaged within the cap, where the first side is adapted for centered engagement in contact with the distal end of the plunger to thereby translate the scalpel within the interior of the cap.

FIG. 12 shows a side perspective enlarged view of the translating hub of FIG. 11 having a lance such as a needle or the depicted scalpel engaged thereon.

### DETAILED DESCRIPTION OF THE INVENTION

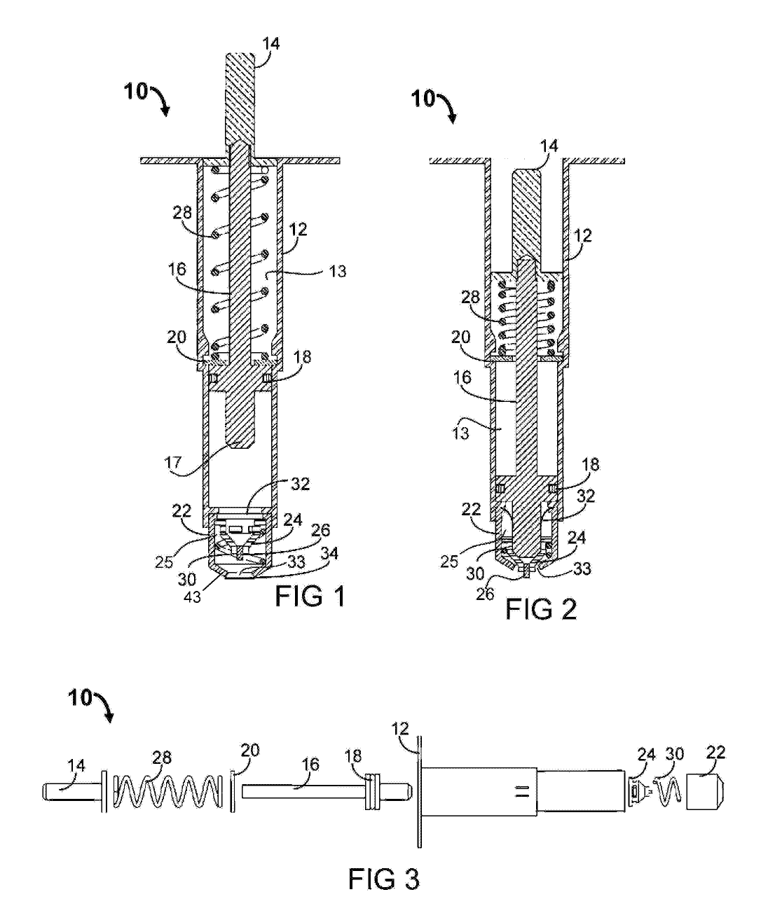

Now referring to drawings in FIGS. 1-12, wherein similar components are identified by like reference numerals, there is seen in FIGS. 1-3, the device 10 configured in one preferred mode adapted to operate as a sterile incising and medicating syringe.

The device includes an actuator 11 configured on a second end to removably engage a cap 22 holding a translatable lance such as a needle or the depicted lance 26. The barrel 12 houses the internal treatment actuation mechanisms, includes cooperative connectors for coupling and decoupling the cap 22 providing the disposable treatment tool.

In preferred modes of the device 10, the barrel 12 should have an axial passage 13 having a diameter and length which are sufficient to operatively house the components housed therein. The actuator 11 at a first end, should form a comfortable grip for the human hand. Such may be for instance two or more perpendicular flanges 15 projecting in opposite directions at the first end of the actuator 11 barrel 12, which define finger holds for leverage and stability during treatment.

The second end of the barrel 12 of the actuator 11, should be configured with a complimentary fastener 23 to that of a fastener located at a first end of the removable cap 22, to securely engage first end of the cap 22 in operative sealed engagement with the second end of the barrel 12 of the actuator 11. Such complimentary fasteners 23 located on both the barrel 12 and cap 22 may be any which would occur to those skilled in the art for such operative engagement. For example such complimentary fasteners 23 can be any from a group of complimentary fasteners including threads, fractional engagements of the first end of the cap 22 with the second end of the barrel 12, bayonet engagements, snaps, or other complimentary fasteners as would occur to those skilled in the art.

The cap 22, preferably configured with upper and outer faces to fasten to the second end of the barrel 12 using complimentary fasteners 23, is sealed prior to this removable engagement to protect the contents housed within an interior chamber 25 of the cap 22 of the device 10 during transportation and storage prior to use.

The hub 24 is slidingly engaged within the interior chamber 25 of the cap 22 on a circumferential edge 43 communicating between a first side of the hub and second side. Preferably, the cap 22 is configured such that a pre-conjoined hub 24 and cap 22 can securely removably engage with the second end of barrel 12 in a manner to perform a single treatment, and then decoupled to be discarded.

As noted the complimentary connectors 23 on the second end of barrel 12 are capable of enduring multiple coupling and decoupling repetitions with successive caps 22 to insurer device 10 longevity. The hub 24 may contain one or an array of small vent holes to permit airflow through the cap 22, during use. As noted above, the interior chamber 25 of the cap 22 can include a reservoir of medical ointment or cream (not shown) to be dispensed from the device 10 through the aperture 33 during treatment, or to just coat and surround a lance 26.

The disposable lance 26 is connected to a center area of the hub 24 which is slidingly engaged within the cap 22 which aligns with the area within the aperture 33 at the second end of the cap 22. When actuated by translation of the plunger 16, the lance 27 engaged with the hub 24 both translate toward the aperture 33 positioned at the second end of the cap 22.

In the favored mode of the device 10, the diameter and edge surface of the aperture 33 is configured to cause a raised portion 27 of the skin to rise into the interior cavity 25 behind the aperture 33. Because of this configuration of aperture 33 causing this raised portion 27 when the cap is compressed against the skin, the lance 26 need not extend out of the aperture 33 in the second end of the cap 22, but need only extend into the interior chamber 25 behind the aperture 33 to contact the raised portion 27 of skin of the user which is drawn therein by pressing the device 10 against the skin.

In another mode of the device 10, where skin might not rise into the aperture 33 and the interior chamber 25 behind it, the lance 26 may be sized to translate to extend slightly from the aperture 33 in and beyond the surface fo the second end of the cap 22. The lance 26 should be sized and sharpened to properly pierce the user's skin during treatment and ideally be limited in total distance of translation. After much experimentation this total translation distance out of the aperture 33 and past the second end of the cap 22 should be less than 2 mm as such as been found to be sufficient to pierce most skin lesions and pustules, but not too long to reach underlying connective tissues and muscles. This distance can be limited by limiting the translation distance of the plunger 16 by having it bottom out on a ledge or washer within the axial passage 13 of the barrel 12.

For increased treatment efficacy and range of uses, the lower face of the cap 22 is configured with a bevel 37 forming an angled circumference of the aperture 33 with a diameter and chamfer sized to effectively draw in an form a raised portion 27 of effected skin area, slightly smaller than the diameter of the aperture. The aperture 33 configuration thus forms the raised portion 27 which extends into the interior chamber 25 slightly, during a compressive engagement of the aperture 33 at the second end of the cap, with the shin or the user. The bevel 37 is preferred as it forms the diameter of the aperture 33 wider as it communicates with the second end of the cap 22, and narrower as it communicates through the endwall 43 at the second end with the interior chamber 25.

After much experimenting with aperture sizes, an aperture between 3 mm to 6 mm has been found to achieve the desired raised portion 27 of skin. A particularly favored diameter of the aperture 33 at its intersection with the second end of the cap exterior surface of the endwall, is 3.5 mm to 4.5 mm in diameter of the aperture 33 has shown to provide an optimal configuration and allow translation of the micro lance 26 to remain within the confines of the cap 22 and still pierce the raised portion 27 of skin.

A first film layer 32 is engaged over the opening 21 on the first end of the cap 22 to maintain sterility during transport. This film layer 32 is formed of material which is pierced by the distal end 17 of the plunger when translated toward the cap 22 when positioned to cover the opening 21 on the first end of the cap 22. Additionally, the angled aperture 33 of the cap 22 should be covered by a removable second film layer 34 to maintain sterility and safety. This second film layer 34 is removed prior to use.

A push knob 14 is positioned at a first end of the plunger 16 and projects from the axial passage 13 of the actuator 11. The plunger 16 in all modes is constrained to slide axially within the axial passage 13 of the barrel 12. The push knob 14 can alternatively fasten to the plunger 16 through one or more of the following rigid and permanent means including but not limited to: magnets, press fit, adhesive, or a flexible coupling, or the push knob 14 and plunger 16 can potentially be combined into a singular component.

The plunger 16, in some modes additionally contains one or more O-rings 18 around its outer face, which are sized and assembled to maintain an airtight seal within the barrel 12, and exhibits a beveled lower face capable of mating with the interior face of the hub 24.

In some modes of the device 10, a captive washer 20 is permanently affixed within the barrel 12. Employing a captive washer 20 increases device 10 durability and reliability by distributing the upper constant force of the compression spring 28 against the thumb knob 14 as it repeatedly contracts and expands. Additionally, in all modes of the device 10, the cap 22 is provided with the hub 24 biased toward the first end of the cap 22 by default. Any biasing component may be employed as would occur to those in the art. Currently a lower compression spring 30, provides a constant biasing force from the second end of the cap 22 toward the first end of the cap 22 which engages the second end of the barrel 12.

Both the upper and lower compression springs 28, and 30, should preferably have closed and grounded ends to reduce wear against its mating components. The cap 22, hub 24, washer 20 and barrel 12 may also contain proper spring interfaces to prevent rotation of the upper and lower compression springs 28 30 and reduce wear.

The biasing components provided by the depicted upper and lower springs 28, 30 can alternatively foe replaced by one or a combination of flexing or biasing mechanisms including but not limited to: air springs, leaf springs, disk springs or flexures.

Employing the device 10 described herein, the user may remove the first film layer 32 from the opening 21 at the first end of the cap 22, or they may keep it in place and allow the distal end 17 of the plunger to simply pierce it during use. The user then removably engages the complimentary fasteners 23 at the first end of the cap 22 and second end of the barrel 12 and thereby removably engages the cap with the barrel 12. Prior to engaging the second end of the cap 22 with the skin, the user removes the second film layer 34 covering the aperture 33.

The user then firmly grabs the outer cylindrical face of the barrel 12 with one hand and presses the beveled or angled edged aperture 33 at the second end of the cap 22 firmly against the skin. This causes the formation of the raised portion 27 which is drawn into the interior chamber 25 through the aperture 33 and positions the lance 26 having a tip thereof, aligned with and adjacent the effected area. As noted this positioning and the shape and size of the aperture 33 is adapted to draw a section of skin to a raised portion 27 projecting into the aperture 33 at the second end of the cap 22. Such was found to eliminate the need to translate the tip of the lance 26 out of the lower end of the cap 22 where it might injure the user or be able to project too far into the skin. This is a preferred mode of the device 10. Further, it would be preferable to render each cap 22 incapable of a second use once the user actuates the plunger to lance a skin area.

Once the device 10 is centered upon the skin area of choice, and preferably the skin is draw into the aperture 33, the user then presses one finger of his or her other hand against the bottom face of each of the perpendicular flanges on the barrel 12, for leverage, and then depresses the thumb knob 14.

Per FIGS. 2, 5, and 8, depressing the thumb knob 14 collapses the upper compression spring 28, and drives the distal end of the plunger 16 down through the axial passage 13 of the barrel 12 to pierce the first film layer 32 if engaged. As noted this action provides the actuation to translate the lance 26, and if located in the cap, expel any ointment through the aperture 33.

During actuation of a cap 22 to translate the lance 26, the plunger then couples with, and translates the hub 24 which collapses the lower compression spring 30, and translates the hub 24 toward the bottom of the cap 22. This action concurrently translates the lance 26 and the tip thereof. As noted if the skin is drawn into the aperture 33 at the second end of the cap, and the interior chamber 25 adjacent it, the tip 49 of the lance 26 need not translate outside the cap 22.

Once user pressure is removed from the thumb knob 14, the upper and lower compression springs 28, 30 expand to bias against the plunger 16 and hub 24 respectively and bias them in a direction away from the second end of the cap 22, to translate to their starting positions.

As an air tight interface preferably exists between the inner wall defining the axial passage 13 of the barrel 12 and the o-ring 18 on the plunger 16. As such suction is generated from the post-incision vertical translation of the plunger 16, and this suction is communicated to the interior chamber 25 and may be employed to draw extra ointment, pus, blood or fluid from the effected area and into the cap 22 for easy disposal. The user can then discard the cap 22 and engage a new one for a subsequent single use. It is recommended that the patient clean the effected area before and after treatment with an antibacterial or alcohol to prevent infection.

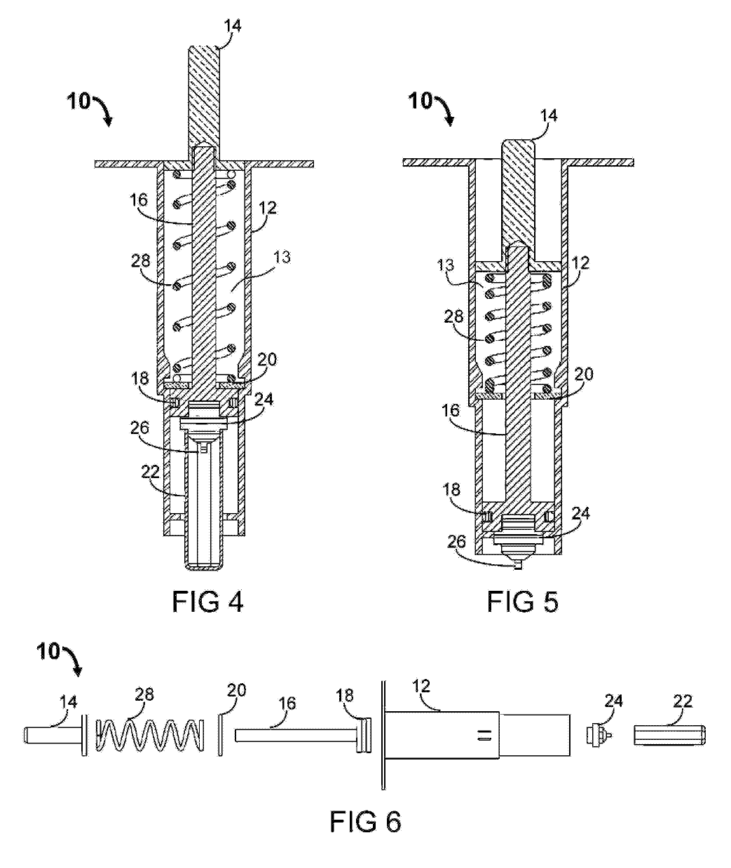

In another preferred node of the device 10, as shown in FIGS. 4-6, the hub 24 may be temporarily coupled directly to the plunger 16, eliminating the requirement for a lower compression spring 30 and any films 32 and 34. The hub 24 and cap 22 may engage with the plunger 16 through matching threads, but can alternatively employ one or a combination of rigid and temporary fastening means including but not limited to: snaps, press fits or keyed grooves.

The cap 22, which protects the user from the lance 26 during transport and use, maintains product sterility as per the first preferred mode of the device 10, also acts as a tool to position the hub 24 for communication with the plunger 16.

The cap 22 and hub 24 should preferably couple through an air tight press fit, whereas three to ten mating faces, with a regular convex polygonic cross section, allows a user to rotate the hub 24 within the barrel 12. The hub 24 and cap 22 can alternatively engage through one or a combination of air tight reversible means including but not limited to: threads, snaps or keyed grooves.

The cap 22, in this second preferred mode of the device 10, should extend through and past the bottom aperture of the barrel 12 to allow the user to engage the hub 24 to the plunger 16 via the cap 22 while the device 10 remains in its expanded configuration.

Additionally, the barrel 12 can be configured in the second preferred mode of the device 10 with a chamfered bottom face to replicate the pinching and skin drawing capabilities of the angled aperture 33 in the cap 22 in the first preferred mode described above. Employing the device 10 herein per its second configuration, the user inserts the preassembled and sealed hub 24 and cap 22 assembly through the lower aperture in the barrel 12, affixes the hub 24 to the plunger 16 via the cap 22, and removes the cap 22.

After proceeding with treatment as described above in the first preferred mode of the device, the user employs the cap 22 to decouple the hub 24 from the plunger 16 and can then discard the cap 22 and hub 24.

The barrel 12, thumb knob 14, plunger, cap 22 and hub 24 can be composed of one or a combination of medical grade and preferably transparent materials including but not limited to: polycarbonate, acrylic, plastic or glass.

One or a combination of non-reactive, medical grade and plastically deforming materials can be used to construct the upper and lower springs 28, 30 including but not limited to: steel, stainless steel, aluminum, plastic, carbon fiber or wood. The containment film 32a, upper sanitary film 32b, and lower sanitary film 34 can be composed of any materials which are typically used as thin covering materials, and may contain medical grade adhesive backing for adhesion to their respective components.

The captive washer 20 can be made of one or a combination of durable medical grade materials including but not limited to: plastic, aluminum, steel, stainless steel, polycarbonate or fiberglass. The o-rings 18 can be composed of any non-reactive medical grade materials commonly used to make in o-rings including but not limited to: silicone or rubber.

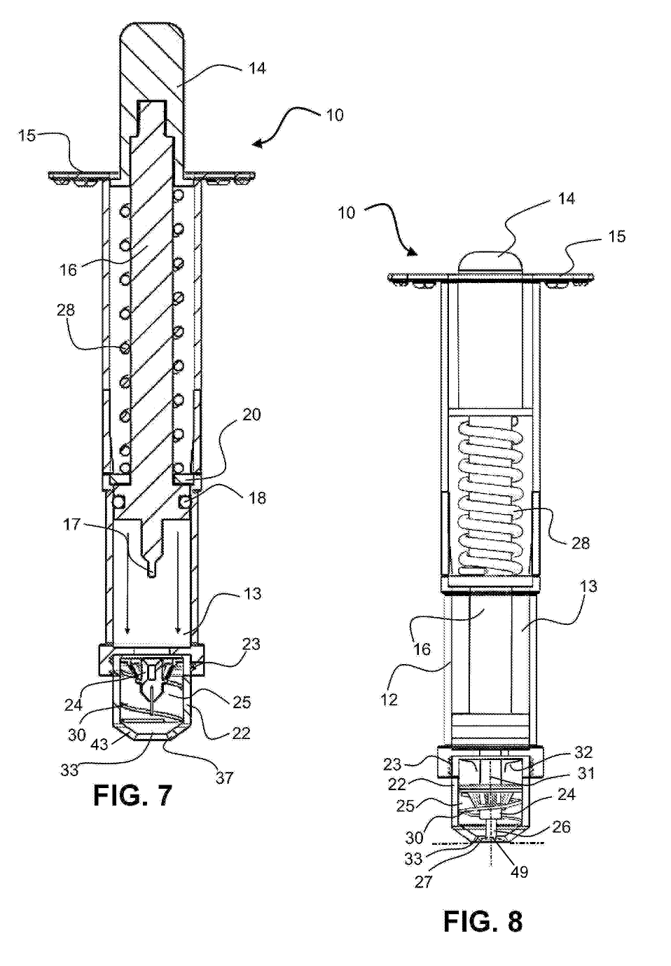

As noted FIGS. 7-12 depict another particularly preferred mode of the device 10 herein. In FIG. 7 is seen the device 10 having an actuator 11 having the plunger 16 within the axial passage 13 of the barrel 12 in the default retracted position, biased thereto by spring 28. Also shown is the removably engageable cap 22 having a lance 26 biased and translated away from the second end of the cap wherein the aperture 33 communicating with the interior chamber 25 is located. As noted this aperture 33 is covered by the second film layer 34 prior to use to maintain sterility.

In the mode of FIGS. 7-12 the distal end 17 of the plunger 16 is formed to a smaller diameter than that of the rest of the plunger 16. This decreased diameter forms a point that easily pierces the first film layer 32 covering the opening 21 of the cap 22 shown in FIG. 10 for example. Additionally, this distal end 17 is preferably formed in shape and size which is complimentary to a recess 29 (FIG. 11) formed on a first side surface of the hub 24. This allows the distal end 17 of the plunger 16 to form an engagement with the recess 29 when translated, which has been found to help maintain the hub 24 centered during travel within the interior chamber 25.

This engagement of distal end 17 and recess 29 is also preferred as it prevents the hub 24 from tilting during travel and maintains the axis of the lance 41, aligned with the axis 31 of the interior chamber 25 and the axis 19 of the axial passage 13, during translation in the interior chamber 25. This is most important to maintain the tip of the lance 26 projecting straight and continuously aligned with the aperture 33 during translation.

Still further, the length of translation of the hub 24 and thus the lance 26, can be adjusted during manufacture, by forming the recess 29 deeper into the first side of the hub 24 or shallower. This causes contact with the distal end 17 of the plunger 16 with the bottom wall of the recess 29, to change and thus the travel of the hub 24 to increase or decrease depending on the depth of the recess 29. This preferred travel distance is noted above.

FIG. 8 shows the device of FIG. 7 moved to the extended position, by translation of the plunger 16 of the actuator 11 in a direction toward the second end of the barrel 12 to which the cap 22 is removably engaged. The distance of translation is preferably such that the lance 26 engaged with the hub 24 does not extend from the aperture 33.

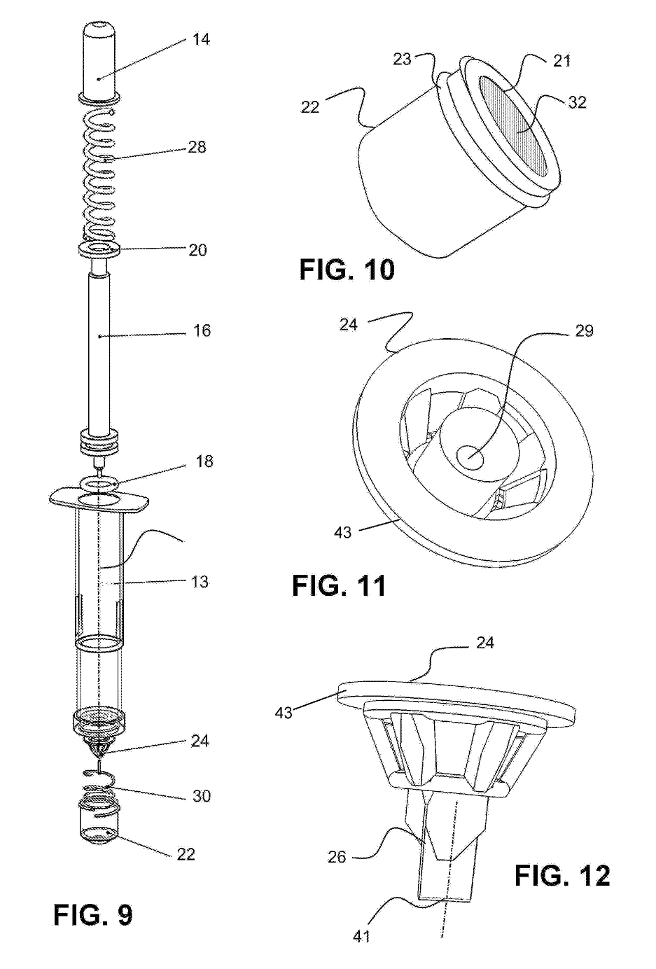

As noted FIG. 9, shows an exploded view of the device 10 depleted in FIGS. 7-8.

Shown in FIG. 10, is the removably engageable cap 22 in which the hub 24 is translatably engaged within. At the first end of the cap 22 is located the complementary fasteners 23 which engage with complimentary fasteners 23 at the second end of the barrel 23. Threads are shown as the complementary fasteners 23 but others may be employed. A sealed removable engagement can thereby be achieved between the cap 22 and the barrel 12. A first film layer 32 is positioned to seal the opening 21 located on the first end of the cap 22.

In FIG. 11 is shown a first side surface of the hub 24 which is slidably engaged within the interior chamber 25 of the cap 22. The circumferential edge of the hub 24 is sized to slidably engage the wall surface forming the interior chamber 25 of the cap 22.

Finally, FIG. 12, shows a side perspective enlarged view of the translating hub 24 of FIG. 11. The lance 26 is located at the second end of the hub 24 and projects along the axis 41 of the hub 24. Currently a scalpel is shown as the lance 26 however other sharp pointed projections may be employed such as needles or the like.

As noted, any of the different configurations and components can be employed with any other configuration or component shown and described herein. Additionally, while the present invention has been described herein with reference to particular embodiments thereof and steps in the method of production, a latitude of modifications, various changes and substitutions are intended in the foregoing disclosures, it will be appreciated that in some instance some features, or configurations, or steps in formation of the invention could be employed without a corresponding use of other features without departing from the scope of the invention as set forth in the following claims. All such changes, alternations and modifications as would occur to those skilled in the art are considered to be within the scope of this invention as broadly defined in the appended claims.

Further, the purpose of any abstract of this specification is to enable the U.S. Patent and Trademark Office, the public generally, and especially the scientists, engineers, and practitioners in the art who are not familiar with patent or legal terms or phraseology, to determine quickly from a cursory inspection the nature and essence of the technical disclosure of the application. Any such abstract is neither intended to define the invention of the application, which is measured by the claims, nor is it intended to be limiting, as to the scope of the invention in any way.

What is claimed is:

1. The syringe actuated lance apparatus, comprising:
an actuator having a barrel with an axial passage running between a first end of said barrel to a second end thereof;
a plunger slidably engaged in said axial passage;
said plunger biased toward said first end of said barrel to a first position locating a distal end of said plunger spaced from said, second end of said barrel;
said second end of said barrel adapted to removably engage a first end of a cap;
an interior chamber communicating with an opening at said first end of said cap and an aperture communicating through an endwall at a second end of said cap;
a lance slidingly engaged within said interior chamber, said lance having a first position, biased toward said first end of said cap with a tip of said lance spaced a first distance from said aperture;
said lance translatable to a second position locating said tip thereof a second distance from said aperture, said second distance being closer to said aperture than said first distance; and
said lance being translatable to said second position by a translation of said plunger toward said second end of said barrel.

2. The syringe actuated lance apparatus of claim 1, additionally comprising:
said endwall at said second end of said cap, configured for a compressive engagement against skin of a user; and
said aperture having a diameter determining a diameter of a raised portion of said skin extending into said interior chamber of said cap during said compressive engagement, whereby said lance translated to said second position with said endwall in said compressive engagement, pierces said raised portion of skin.

3. The syringe actuated lance apparatus of claim 1, additionally comprising:
a hub, said hub having a first side and a second side and having a circumferential edge communicating therebetween;
said circumferential edge in a sliding engagement with sidewalls of said interior chamber;
said lance in an engagement at one end with said second side of said hub, said lance extending from said engagement to said tip of said lance.

4. The syringe actuated lance apparatus of claim 2, additionally comprising:
a hub, said hub having a first side and a second side and having a circumferential edge communicating therebetween;
said circumferential edge in a sliding engagement with sidewalls of said interior chamber;
said lance in an engagement at one end with said second side of said hub, said lance extending from said engagement to said tip of said lance.

5. The syringe actuated lance apparatus of claim 3, additionally comprising:
a recess depending into said first side of said hub; and
said distal end of said plunger sized for an engagement within said recess upon said translation of said plunger toward said second end of said barrel; and
said engagement of said distal end of said plunger with said recess defining a supportive connection to maintain an axis of said lance aligned with a central axis running through said interior chamber between said opening and said aperture.

6. The syringe actuated lance apparatus of claim 4, additionally comprising:
a recess depending into said first side of said hub; and
said distal end of said plunger sized for an engagement within said recess upon said translation of said plunger toward said second end of said barrel; and
said engagement of said distal end of said plunger with said recess defining a supportive connection to maintain an axis of said lance aligned with a central axis running through said interior chamber between said opening and said aperture.

7. The syringe activated lance apparatus of claim 3, additionally comprising:
a first film layer covering said opening on said first side thereof; and
a second film layer covering said aperture in said endwall.

8. The syringe actuated lance apparatus of claim 4, additionally comprising:
a first film layer covering said opening on said first side thereof; and
a second film layer covering said aperture in said endwall.

9. The syringe actuated lance apparatus of claim 5, additionally comprising:
a first film layer covering said opening on said first side thereof; and
a second film layer covering said aperture in said endwall.

10. The syringe actuated lance apparatus of claim 6, additionally comprising:
a first film layer covering said opening on said first side thereof; and
a second film layer covering said aperture in said endwall.

11. The syringe actuated lance apparatus of claim 1, additionally comprising:
said aperture communicating through an endwall at a second end of said cap having a beveled edge; and
said aperture having a diameter narrowing from a wider diameter where said aperture meets an exterior surface of said endwall, to a narrower diameter where said aperture meets said interior chamber of said cap.

12. The syringe actuated lance apparatus of claim 2, additionally comprising:
said aperture communicating through an endwall at a second end of said cap having a beveled edge; and
said aperture having a diameter narrowing from a wider diameter where said aperture meets an exterior surface of said endwall, to a narrower diameter where said aperture meets said interior chamber of said cap.

13. The syringe actuated lance apparatus of claim 3, additionally comprising:
said aperture communicating through an endwall at a second end of said cap having a beveled edge; and
said aperture having a diameter narrowing from a wider diameter where said aperture meets an exterior surface of said endwall, to a narrower diameter where said aperture meets said interior chamber of said cap.

14. The syringe actuated lance apparatus of claim 4, additionally comprising:
said aperture communicating through an endwall at a second end of said cap having a beveled edge; and
said aperture having a diameter narrowing from a wider diameter where said aperture meets an exterior surface of said endwall, to a narrower diameter where said aperture meets said interior chamber of said cap.

15. The syringe actuated lance apparatus of claim 5, additionally comprising:
said aperture communicating through an endwall at a second end of said cap having a beveled edge; and
said aperture having a diameter narrowing from a wider diameter where said aperture meets an exterior surface of said endwall, to a narrower diameter where said aperture meets said interior chamber of said cap.

16. The syringe actuated lance apparatus of claim 6, additionally comprising:
said aperture communicating through an endwall at a second end of said cap having a beveled edge; and
said aperture having a diameter narrowing from a wider diameter where said aperture meets an exterior surface of said endwall, to a narrower diameter where said aperture meets said interior chamber of said cap.

17. The syringe actuated lance apparatus of claim 1, additionally comprising:
said aperture communicating through an endwall at a second end of said cap having a diameter sized between 3 mm to 6 mm.

18. The syringe actuated lance apparatus of claim 12, additionally comprising:
said wider diameter of said aperture being sized between 3 mm to 6 mm.

19. The syringe actuated lance apparatus of claim 13, additionally comprising:
said wider diameter of said aperture being sized between 3 mm to 6 mm.
